(19) United States
(12) Patent Application Publication
(10) Pub. No.: US 2025/0115852 A1
(43) Pub. Date: Apr. 10, 2025

(54) CELL HANDLING SYSTEM, CELL HANDLING APPARATUS, AND CELL HANDLING CARTRIDGE

(71) Applicants: CANON KABUSHIKI KAISHA, Tokyo (JP); Canon Medical Systems Corporation, Otawara-shi (JP)

(72) Inventors: Kazutaka YANAGITA, Yokohama (JP); Tetsuya SAKAI, Ryugasaki (JP); Akane KUBOYAMA, Kawasaki (JP)

(73) Assignees: CANON KABUSHIKI KAISHA, Tokyo (JP); Canon Medical Systems Corporation, Otawara-shi (JP)

(21) Appl. No.: 18/905,967

(22) Filed: Oct. 3, 2024

(30) Foreign Application Priority Data
Oct. 6, 2023 (JP) 2023-174698

(51) Int. Cl. C12M 1/00; C12M 3/00

(52) U.S. Cl. CPC C12M 29/14; C12M 23/40; C12M 23/42; C12M 23/58

(57) ABSTRACT

A cell handling system includes: a cell handling cartridge including a passage and an inlet, the passage configured to allow a liquid to flow through the passage, the inlet provided to the passage, and configured to allow a gas to pass through the inlet; a body apparatus including a connection portion and a presser, the connection portion being connected to the inlet and having an air passage configured to allow the gas to flow through the air passage, the presser being configured to press the connection portion against the cell handling cartridge; and a seal member on at least one of an opposing face of the connection portion or an opposing face of the cell handling cartridge, the opposing face of the connection portion facing the cell handling cartridge, the opposing face of the cell handling cartridge facing the connection portion.

### CROSS-REFERENCE TO RELATED APPLICATIONS

This application is based upon and claims the benefit of priority from Japanese Patent Application No. 2023-174698, filed Oct. 6, 2023, the entire contents of which are incorporated herein by reference.

### FIELD

Embodiments described herein relate generally to a cell handling system, a cell handling apparatus, and a cell handling cartridge.

### BACKGROUND

The preparation of an induced pluripotent stem cell (hereinafter referred to as an “iPS cell”) is performed manually by an operator with skills using equipment such as a safety cabinet. Thus, the preparation of an iPS cell requires considerable expense. This has been one of the obstacles to clinical application. Also, there are apparatuses such as a closed cell preparation apparatus that automates performance of predetermined processes such as cell culturing and a cell processing apparatus that allows a user to set any process. Even if such apparatuses are used, the preparation of an iPS cell requires equipment such as a safety cabinet.

In order to prepare an iPS cell automatically and at a low cost, the use of a system that combines a closed cartridge that incorporates a passage for delivering a cell suspension and a drive mechanism that drives liquid delivery in the cartridge can be considered. As a method of driving liquid delivery, an air pressure feeding method, for example, can be used.

If the air pressure feeding method is adopted, it is necessary to fit and connect a cartridge and an air pressure feeding mechanism to each other. Also, when connecting the cartridge and the air pressure feeding mechanism to each other, it is necessary to secure airtightness, thus requiring precise molding of a connection portion and precise positioning at the time of the connection, causing increased apparatus manufacturing costs.

### BRIEF DESCRIPTION OF THE DRAWINGS

FIG. 1 is a diagram showing a configuration of a cell handling system according to a first embodiment.

FIG. 2 is a diagram showing an example of a configuration of a cell handling mechanism according to the first embodiment.

FIG. 3 is a view of the cell handling mechanism shown in FIG. 2 from one side in a Y-axis direction.

FIG. 4 is a view of the cell handling mechanism shown in FIG. 2 from one side in an X-axis direction.

FIG. 5 is an enlarged view of the connection portion of the pressure feeding pad and the inlet shown in FIG. 3.

FIG. 6 is a flowchart illustrating a procedure of liquid delivery performed by the system according to the first embodiment.

FIG. 7 is a view of a state in which the pressure feeding pad has moved to above the inlet from the state shown in FIG. 3.

FIG. 8 is a view of a state in which the pressure feeding pad has descended from the state shown in FIG. 7.

FIG. 9 is a view of the cell handling mechanism shown in FIG. 8 from one side in an X-axis direction.

FIG. 10 is an enlarged view of the connection portion of the pressure feeding pad and the inlet shown in FIG. 8.

FIG. 11 is an enlarged view of the connection portion of a pressure feeding pad and an inlet according to a first modification.

FIG. 12 is a view of a state in which the pressure feeding pad has descended from the state shown in FIG. 11.

FIG. 13 is an enlarged view of the connection portion of a pressure feeding pad and an inlet according to a second modification.

FIG. 14 is a view of a state in which the pressure feeding pad has descended from the state shown in FIG. 13.

FIG. 15 is an enlarged view of the connection portion of a pressure feeding pad and an inlet according to a third modification.

FIG. 16 is a view of a state in which the pressure feeding pad has descended from the state shown in FIG. 15.

FIG. 17 is an enlarged view of the connection portion of a pressure feeding pad and an inlet according to a fourth modification.

FIG. 18 is a view of a state in which the pressure feeding pad has descended from the state shown in FIG. 17.

FIG. 19 is an enlarged view of the connection portion of a pressure feeding pad and an inlet according to a fifth modification.

FIG. 20 is a view of a state in which the pressure feeding pad has descended from the state shown in FIG. 19.

FIG. 21 is a view of a cell handling mechanism according to a second embodiment from one side in a Y-axis direction.

FIG. 22 is a view of the cell handling mechanism according to the second embodiment from one side in an X-axis direction.

FIG. 23 is a flowchart illustrating a procedure of liquid delivery performed by a system according to the second embodiment.

FIG. 24 is a view of a state in which the pressure feeding pad has moved to above the inlet from the state shown in FIG. 21.

FIG. 25 is a view of a state in which the pressure feeding pad has descended from the state shown in FIG. 24.

FIG. 26 is a view of the cell handling mechanism shown in FIG. 25 from one side in an X-axis direction.

FIG. 27 is a view of a cell handling mechanism according to a third embodiment from one side in an X-axis direction.

FIG. 28 is a view of a state in which a cylinder shaft is fixed by a fixing mechanism, shifted from the state shown in FIG. 27.

FIG. 29 is a flowchart illustrating a procedure of liquid delivery performed by a system according to the third embodiment.

FIG. 30 is a view of a cell handling mechanism according to a modification of the third embodiment from one side in an X-axis direction.

FIG. 31 is a view of a state in which a cylinder shaft is fixed by a fixing mechanism, shifted from the state shown in FIG. 30.

### DETAILED DESCRIPTION

In general, according to one embodiment, a cell handling system includes: a cell handling cartridge including a passage and an inlet, the passage configured to allow a liquid used for cell handling to flow through the passage, the inlet provided to the passage, opened toward outside, and configured to allow a gas used for delivery of the liquid to pass through the inlet; a body apparatus including a connection portion and a presser, the connection portion being connected to the inlet and having an air passage configured to allow the gas to flow through the air passage, the presser being configured to press the connection portion against the cell handling cartridge; and a seal member on at least one of an opposing face of the connection portion or an opposing face of the cell handling cartridge, the opposing face of the connection portion facing the cell handling cartridge, the opposing face of the cell handling cartridge facing the connection portion.

Hereinafter, a cell handling system will be described with reference to the accompanying drawings. In the description below, constituents having substantially the same functions and configurations will be denoted by the same reference symbols, and a repeat description of such constituents will be given only where necessary.

### First Embodiment

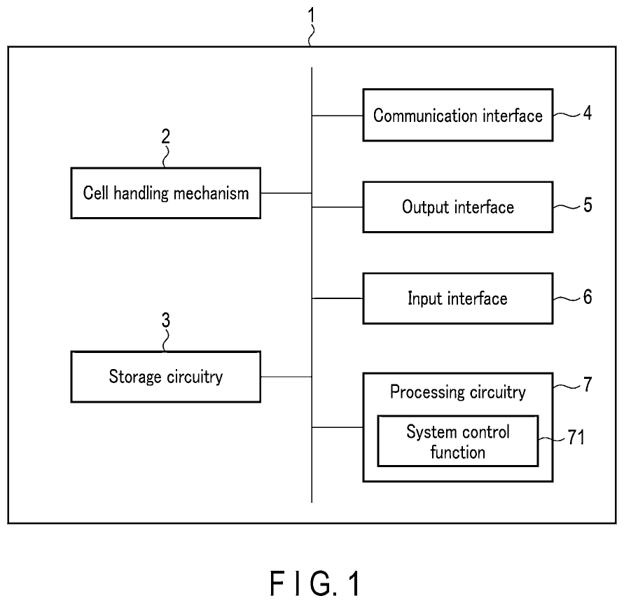

FIG. 1 is a block diagram showing an example of a configuration of a cell handling system 1 according to a first embodiment. The cell handling system 1 is a system for automatically performing cell handling. The cell handling includes, for example, a process of preparing an iPS cell (hereinafter referred to as a “cell preparation process”) and/or a process of processing a cell (hereinafter referred to as a “cell processing process”). The description below introduces a case of preparing and/or processing an iPS cell as an example; however, the cell handling system 1 can be used not only to prepare and/or process an iPS cell but also to prepare and/or process any cells.

The cell preparation process or the cell processing process includes, for example, a process of separating blood cells in the blood to extract necessary cells, a process of introducing a factor into a peripheral blood mononuclear cell (hereinafter referred to as a “PBMC”) after expansion culture, a process of culturing a cell, etc. The cell handling system 1 may perform all or only some of the aforementioned processes. The cell handling system 1 may be connected to an external network or database via a network.

As shown in FIG. 1, the cell handling system 1 includes a cell handling mechanism 2, storage circuitry 3, a communication interface 4, an output interface 5, an input interface 6, and processing circuitry 7.

The cell handling mechanism 2 is a mechanism for performing cell handling. A detailed configuration of the cell handling mechanism 2 will be described later.

The storage circuitry 3 stores programs to be executed by the processing circuitry 7, various types of data to be used in the processing performed by the processing circuitry 7, and the like. Such programs include, for example, a program that is pre-installed in a computer via a network or a non-transitory computer-readable storage medium to cause the computer to implement each function of the processing circuitry 7. The various types of data as used herein are typically digital data. The storage circuitry 3 is an example of a storage.

The communication interface 4 is an interface for performing data communication with an external device via a network.

The output interface 5 is connected to the processing circuitry 7 and outputs a signal supplied from the processing circuitry 7. The output interface 5 is realized, for example, by display circuitry, print circuitry, a voice device, and the like. The display circuitry includes, for example, a CRT display, a liquid crystal display, an organic EL display, an LED display, a plasma display, and the like. The display circuitry may also be processing circuitry that converts data showing a display target into a video signal and outputs the video signal to an external device. The print circuitry includes, for example, a printer and the like. The print circuitry may also be output circuitry that outputs data showing a print target to an external device. The voice device includes, for example, a speaker and the like. The voice device may be output circuitry that outputs a voice signal to an external device.

The input interface 6 receives, for example, various operations from an operator. The input interface 6 is realized by, for example, a mouse, a keyboard, a touch pad which allows input of instructions through a touch on its operation screen, etc. The input interface 6 is connected to the processing circuitry 7, thereby converting an operational command that is input by the operator into an electric signal and outputting the electric signal to the processing circuitry 7.

The input interface 6 is not limited to one that includes physical operational components such as a mouse and a keyboard. For example, the input interface 6 may be processing circuitry that receives an electric signal corresponding to an operation command input from an external input device provided separately from the cell handling system 1 and outputs the electric signal to the processing circuitry 7.

The processing circuitry 7 is a processor that functions as the center of the cell handling system 1. The processing circuitry 7 performs a system control function 71 by executing a program read from the storage circuitry 3. With the system control function 71, the processing circuitry 7 comprehensively controls each component of the cell handling system 1. For example, in the system control function 71, the processing circuitry 7 controls the driving of each element of the cell handling mechanism 2 for the implementation of the liquid delivery described later. The processing circuitry 7 that implements the system control function 71 is an example of a controller.

The terminology “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in the storage circuitry 3. On the other hand, if the processor is an ASIC, its functions are directly incorporated into the circuitry of the processor as logic circuitry, instead of being incorporated into a program being stored in the storage circuitry 3. Each processor of the present embodiment is not limited to being configured as single circuitry; multiple sets of independent circuitry may be integrated into a single processor that implements its functions. Furthermore, the plurality of components in FIG. 1 may be integrated into a single processor to implement the functions of the processor. The above description of the “processor” also applies to each embodiment and modification described below.

In the present embodiment, descriptions will be given on the premise that each function is implemented by a single processor; however, the embodiment is not limited thereto. For example, a plurality of independent processors may be combined to constitute control circuitry, and the respective processors may implement the respective functions by executing the programs. The system control function 71 may be referred to as system control circuitry or installed as individual hardware circuitry. The above description of the respective functions implemented by the processing circuitry 7 also applies to each embodiment and modification described below. The processing circuitry 7 may include a storage area for storing at least part of the data stored in the storage circuitry 3.

Next, a configuration of the cell handling mechanism 2 will be described in detail.

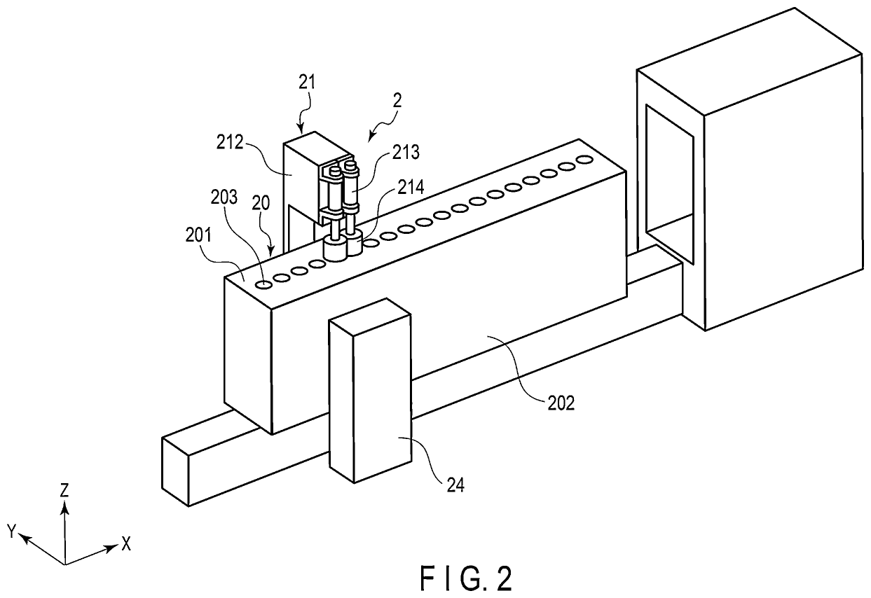

FIG. 2 is a schematic diagram showing an example of a configuration of the cell handling mechanism 2. The cell handling mechanism 2 is a device for performing cell handling using a cell handling cartridge 20. As shown in FIG. 2, the cell handling mechanism 2 includes a liquid delivery mechanism 21 and a valve open-close mechanism 24. The cell handling system 1 is constituted by the cell handling cartridge 20 and a body apparatus including components except the cell handling cartridge 20. The body apparatus is an example of a cell handling apparatus.

The cell handling cartridge 20 is a closed cartridge for automatically performing cell handling. The cell handling cartridge 20 performs one or more processes among the processes included in the cell preparation process or the cell processing process. The cell handling cartridge 20 is attached in a detachable fashion, and multiple types of cell handling cartridges 20 that perform different processes can be replaced with each other and can be attached to be used. To prevent infection, the cell handling cartridge 20 is discarded after use. Multiple cell handling cartridges 20 may be attached to the cell handling mechanism 2.

The cell handling cartridge 20 includes one or more closed passages and containers in the cell handling cartridge 20. Typically, the cell handling cartridge 20 includes multiple passages and multiple containers in the cell handling cartridge 20. Each passage is a duct line for transferring a liquid including a reagent or a cell used for cell handling, and is formed of a tube-shaped resin or plastic. Each container is formed of a resin or plastic. For example, polycarbonate (PC) or polystyrene (PS) is used as a resin.

A plurality of inlets 203 provided to the respective passages and opened to the outside are formed on an upper surface 201 of the cell handling cartridge 20. The inlets 203 are openings. The liquid delivery mechanism 21 is connected to each of the inlets 203. In the liquid delivery process, a gas used for air pressure feeding passes through the inlets 203. The liquid delivery process includes, for example, a process of transferring a liquid collected after the processing is performed to the next processing position, a process of transferring a cell liquid to pass the cell liquid through a filter, and a process of transferring a liquid to an ejection port to diffuse the liquid from the ejection port. The liquid to be transferred may be blood, a blood suspension, a liquid containing PBMCs, a liquid containing cells such as a culture liquid, or a liquid used for cell processing such as a reagent, medium, or factor.

Here, a direction coinciding with a longitudinal direction of the cell handling cartridge 20 in the horizontal direction is defined as an X-axis direction, and a depth direction of the cell handling cartridge 20 in the horizontal direction is defined as a Y-axis direction. The X-axis direction and the Y-axis direction are orthogonal to each other. Also, a height direction of the cell handling cartridge 20 is defined as a Z-axis direction. The Z-axis direction coincides with the vertical direction and is orthogonal to each of the X-axis direction and the Y-axis direction.

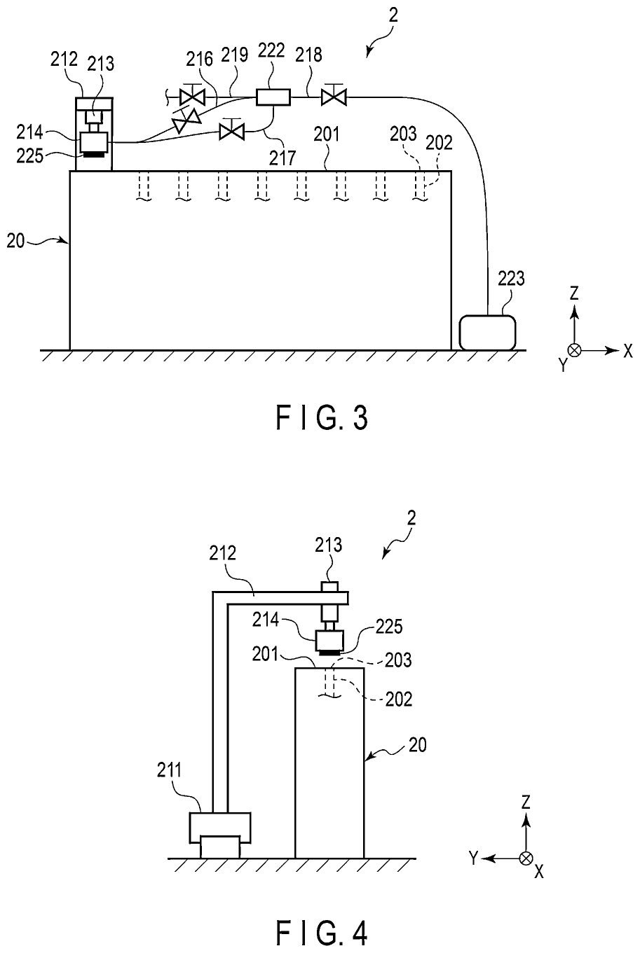

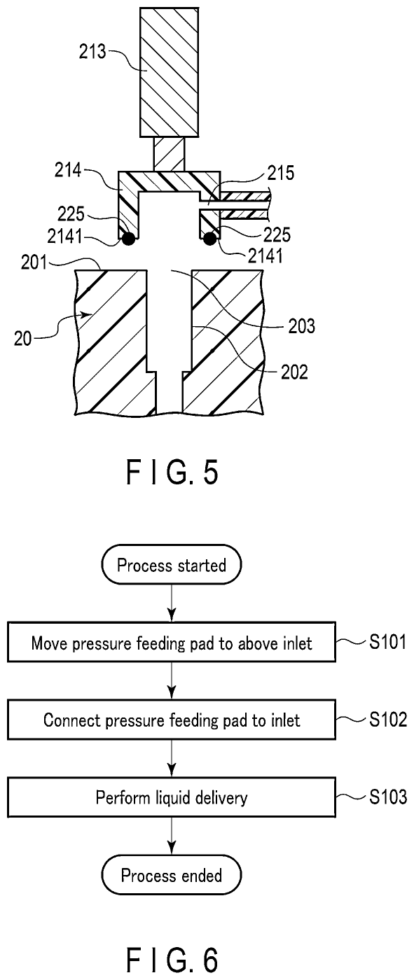

FIG. 3 is a view of the cell handling mechanism 2 from one side in the Y-axis direction. FIG. 4 is a view of the cell handling mechanism 2 from one side in the X-axis direction. FIG. 5 is an enlarged cross-sectional view of the connection portion of the liquid delivery mechanism 21 and the cell handling cartridge 20.

An air filter is provided to each inlet 203. The air filter is a filter for separating the inside and the outside of the cell handling cartridge 20 so as to allow gas replacement. For example, a sheet-shape sterile filter can be used as the air filter. With the air filter provided, it is possible to prevent the liquid inside the cell handling cartridge 20 from flowing out to the outside and to prevent bacteria from flowing into the cell handling cartridge 20 from the outside.

The liquid delivery mechanism 21 is connected to the cell handling cartridge 20 and delivers a liquid using compressed air. Under the control of the processing circuitry 7, the liquid delivery mechanism 21 transfers a liquid inside the cell handling cartridge 20 by a pressure feeding method utilizing compressed air. The liquid delivery mechanism 21 may be referred to as an air pressure feeding mechanism, a pneumatic device, or a pneumatic control device.

The valve open-close mechanism 24 can move inside the cell handling mechanism 2. Under the control of the processing circuitry 7, the valve open-close mechanism 24 opens and closes the valve in the cell handling cartridge 20 and switches the passages in the cell handling cartridge 20. The valve open-close mechanism 24 opens and closes the valve in the cell handling cartridge 20 without touching it using, for example, a magnet. The valve open-close mechanism 24 is an example of a passage switching device.

As shown in FIGS. 2 to 5, the liquid delivery mechanism 21 includes a support 211, an arm 212, an air cylinder 213, a pressure feeding pad 214, an air supply port 215, a first duct line 216 to a fourth duct line 219, an ejector 222, a compressor 223, and a seal member 225.

The support 211 is arranged on the bottom surface of the liquid delivery mechanism 21 and supports the arm 212. The arm 212 fixes the air cylinder 213 in a position above the cell handling cartridge 20. The pressure feeding pad 214 is fixed to a lower end of the air cylinder 213.

The support 211 is a robot that can move on the floor surface of the liquid delivery mechanism 21 in the X-axis direction. The support 211 is a moving mechanism that moves the arm 212, the air cylinder 213, and the pressure feeding pad 214 along the X-axis direction under the control of the processing circuitry 7.

The pressure feeding pad 214 has an air passage through which a gas used for liquid delivery flows and is a connection mechanism for connecting the liquid delivery mechanism 21 with the cell handling cartridge 20 in an airtight state. The pressure feeding pad 214 is an example of a connection portion. Under the control of the processing circuitry 7, the air cylinder 213 moves the pressure feeding pad 214 with respect to the Z-axis direction. Under the control of the processing circuitry 7, the air cylinder 213 presses the pressure feeding pad 214 against the cell handling cartridge 20 and presses the pressure feeding pad 214 against the upper surface 201 of the cell handling cartridge 20, thereby connecting the pressure feeding pad 214 with the passage 202 in the cell handling cartridge 20. The air cylinder 213 is an example of a presser.

The pressure feeding pad 214 is provided with the air supply port 215. The air supply port 215 is an opening. The ejector 222 is connected to the air supply port 215 via the first duct line 216 and the second duct line 217. The ejector 222 is connected to the compressor 223 via the third duct line 218. The ejector 222 is also connected to an exhaust port of the cell handling system 1 or to an exhaust passage in an installation chamber of the cell handling mechanism 2 via the fourth duct line 219. The fourth duct line 219 may be connected to the outside of the chamber via a filter and discharge, to the environment, a gas that has passed through the filter. Each of the first duct line 216 to the fourth duct line 219 is provided with a valve that switches between open and closed states. Under the control of the processing circuitry 7, each valve is switched between open and closed states.

Under the control of the processing circuitry 7, the ejector 222 depressurizes the passage 202 in the cell handling cartridge 20 via the pressure feeding pad 214 and suctions the gas in the passage 202 in the cell handling cartridge 20. The ejector 222 discharges the suctioned gas via the fourth duct line 219. Under the control of the processing circuitry 7, the compressor 223 pressurizes the passage 202 in the cell handling cartridge 20 via the pressure feeding pad 214.

A pressure feeding device for pressure-feeding the liquid in the cell handling cartridge 20 is formed by the pressure feeding pad 214, the ejector 222, the compressor 223, the first duct line 216 to the fourth duct line 219, and the valves of the first duct line 216 to the fourth duct line 219. The processing circuitry 7 controls the driving of the air cylinder 213 and the pressure feeding device and causes the liquid delivery to be performed by the pressure feeding device in the state where the pressure feeding pad 214 is pressed by the air cylinder 213. The processing circuitry 7 also controls the magnitude of the force with which the connection portion presses the cell handling cartridge 20 by controlling the force with which the air cylinder 213 presses the pressure feeding pad 214. Specifically, by controlling the cylinder pressure of the air cylinder 213, the processing circuitry 7 controls the magnitude of the force with which the air cylinder 213 presses the pressure feeding pad 214 against the cell handling cartridge 20.

As shown in FIG. 5, the pressure feeding pad 214 includes an opposing face 2141 that faces the upper surface 201 of the cell handling cartridge 20 when the pressure feeding pad 214 is connected to the cell handling cartridge 20. The opposing face 2141 is a face perpendicular to the direction of pressing by the air cylinder 213. In the present embodiment, the opposing face 2141 is a plane facing vertically downward. The upper surface 201 of the cell handling cartridge 20 is an opposing face that faces the opposing face 2141 of the pressure feeding pad 214 when the cell handling cartridge 20 is connected to the pressure feeding pad 214. The upper surface 201 is a face perpendicular to the direction of pressing by the air cylinder 213. In the present embodiment, the upper surface 201 is a plane facing vertically upward. An opening through which a gas used for liquid delivery passes is formed in the opposing face 2141. The diameter of the inlet 203 of the cell handling cartridge 20 is formed to be smaller than the diameter of the opening formed in the opposing face 2141.

The seal member 225 is attached to the opposing face 2141 of the pressure feeding pad 214. The seal member 225 is a sealer provided to connect the pressure feeding pad 214 with the cell handling cartridge 20 in an airtight state. It suffices that the seal member 225 can connect the pressure feeding pad 214 with the cell handling cartridge 20 in an airtight state. For example, an elastomer is used for the seal member 225. The seal member 225 is preferably placed around the opening formed in the opposing face 2141 so as to surround the opening. For example, an O-ring can be used as the seal member 225. The seal member 225 is preferably formed on a part of the opposing face 2141 rather than on the entire opposing face 2141. This is because the seal member 225 is crushed and the opposing face 2141 comes into contact with the upper surface 201 of the cell handling cartridge 20 when the opposing face 2141 is pressed against the upper surface 201 by the air cylinder 213, thereby allowing maintenance of higher airtightness.

Next, an operation of the cell handling system 1 of the present embodiment will be described.

FIG. 6 is a flowchart showing an example of a procedure of liquid delivery performed by the cell handling system 1. The liquid delivery is performed by the processing circuitry 7 when the liquid delivery process in the cell preparation process or the cell processing process is performed. The liquid delivery process is, for example, one of the series of processes for performing cell handling. The procedure of each step described below is a mere example, and each step can be altered as appropriate to the extent possible. Omission, replacement, and addition of a step can be made as appropriate in the procedure explained below according to the manner in which the embodiment will be implemented. The foregoing explanation of the procedure of each step applies to each of the embodiments and modifications described below. The driving of the respective configurations described below is performed under the control of the processing circuitry 7, a description of which is omitted.

As shown in FIG. 3, the liquid delivery is started in a state where the cell handling cartridge 20 is attached to the cell handling mechanism 2 and the support 211, the arm 212, the air cylinder 213, and the pressure feeding pad 214 are in an initial position.

### Step S101

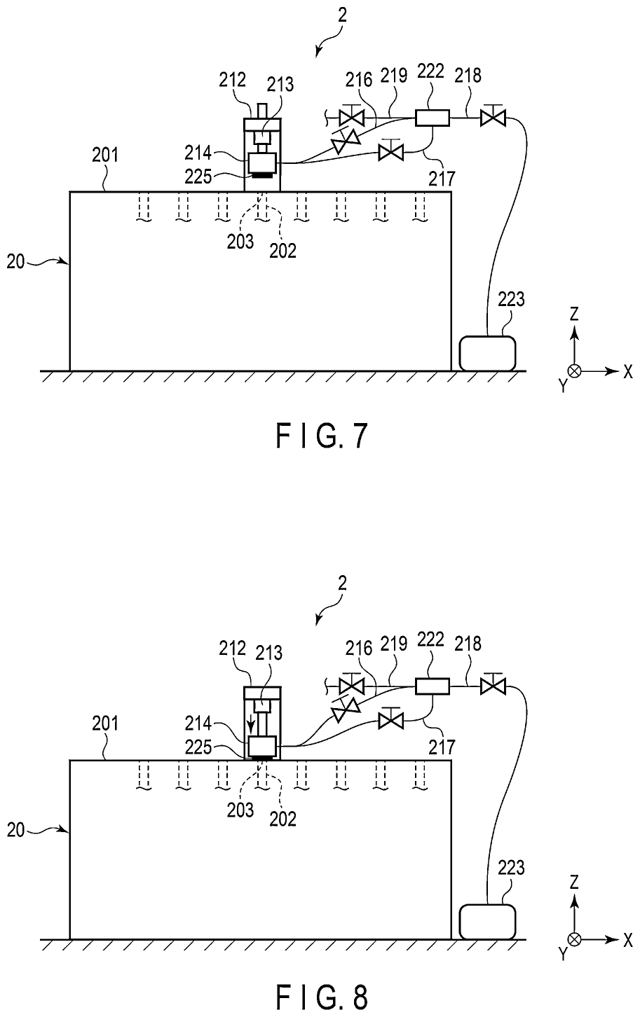

In the liquid delivery, positioning of the pressure feeding pad 214 and the cell handling cartridge 20 with respect to the horizontal direction is performed first. At this time, the support 211 moves the pressure feeding pad 214 along the X-axis direction such that an opening portion defining the opening of the pressure feeding pad 214 is positioned above the inlet 203 through which liquid delivery is performed. FIG. 7 is a view of a state in which the pressure feeding pad 214 has moved to above the inlet 203 through which liquid delivery is performed. FIG. 7 is a view of the cell handling mechanism 2 from one side in the Y-axis direction.

### Step S102

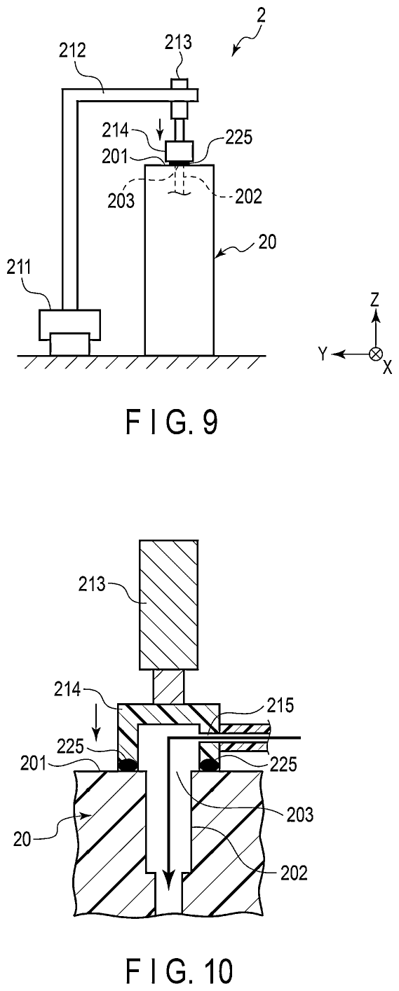

Next, the air cylinder 213 lowers the pressure feeding pad 214, and presses the pressure feeding pad 214 against the upper surface 201 of the cell handling cartridge 20 such that the opening portion of the pressure feeding pad 214 covers the inlet 203 through which liquid delivery is performed. FIGS. 8 and 9 show the state in which the pressure feeding pad 214 is pressed against the cell handling cartridge 20. FIG. 8 is a view of the cell handling mechanism 2 from one side in the Y-axis direction. FIG. 9 is a view of the cell handling mechanism 2 from one side in the X-axis direction. FIG. 10 is an enlarged cross-sectional view of the connection portion of the liquid delivery mechanism 21 and the cell handling cartridge 20 shown in FIGS. 8 and 9.

As shown in FIGS. 8 to 10, in the state where the pressure feeding pad 214 is pressed against the cell handling cartridge 20, the seal member 225 is pressed from the pressure feeding pad 214 side and crushed between the upper surface 201 of the cell handling cartridge 20 and the opposing face 2141 of the pressure feeding pad 214. Thus, the pressure feeding pad 214 and the inlet 203 of the cell handling cartridge 20 are connected to each other in an airtight state.

### Step S103

When the pressure feeding pad 214 is connected to the cell handling cartridge 20, the liquid delivery mechanism 21 performs liquid delivery via the inlet 203 and the air supply port 215. At the time of the liquid delivery, depressurization is performed by the ejector 222 first with the valve of the first duct line 216 closed and the valves of the second duct line 217, the third duct line 218, and the fourth duct line 219 opened. Thus, a liquid to be delivered is suctioned into the passage 202. Thereafter, the valve of the first duct line 216 is opened, and the valves of the second duct line 217 and the fourth duct line 219 are closed. Then, pressurization is performed by the compressor 223 with the valves of the first duct line 216 and the third duct line 218 opened and the valves of the second duct line 217 and the fourth duct line 219 closed. Thus, the liquid suctioned into the passage 202 is ejected from the passage 202 to a container as a transfer destination.

When the liquid delivery is completed, the air cylinder 213 raises the pressure feeding pad 214 and removes the pressure feeding pad 214 from the inlet 203. Thereafter, the support 211 moves the pressure feeding pad 214 along the X-axis direction to above the inlet 203 with which the next liquid delivery is performed.

Hereinafter, the effects of the cell handling system 1 according to the present embodiment will be described.

The cell handling system 1 of the present embodiment includes the cell handling cartridge 20, the cell handling mechanism 2, and the seal member 225. The cell handling cartridge 20 has the passages 202 and the inlets 203. A liquid used for cell handling flows through the passages 202. The inlets 203 are opened in the passages 202 toward the outside and a gas used for liquid delivery passes through the inlets 203. The cell handling mechanism 2 includes the pressure feeding pad 214 and the air cylinder 213. The pressure feeding pad 214 is an example of a connection portion that is connected to the inlets 203 and has an air passage through which a gas flows. The air cylinder 213 is an example of a presser that presses the pressure feeding pad 214 against the cell handling cartridge.

Also, in the present embodiment, the seal member 225 is attached to the opposing face 2141 of the pressure feeding pad 214 that faces the cell handling cartridge 20. The seal member 225 is an example of a sealer. An elastomer other than the seal member 225 may be used as a sealer.

In general, to connect the pressure feeding pad with the inlet, an inlet formed in a tubular shape is inserted into the opening portion of the pressure feeding pad, followed by sealing using an O-ring arranged inside the pressure feeding pad. In this case, to connect the pressure feeding pad with the inlet, high positioning accuracy is required when moving the pressure feeding pad. High molding accuracy is also required since the inlet of the cartridge needs to be formed in a tubular shape having an outer diameter matching an O-ring. This requires a large-scale molding apparatus, leading to increased cartridge manufacturing costs.

By contrast, according to the cell handling system 1 of the present embodiment, the seal member 225 is on the opposing face 2141 that faces the cell handling cartridge 20, and the seal member 225 is pressed against and brought into contact with the cell handling cartridge 20, whereby the cell handling cartridge 20 and the liquid delivery mechanism 21 as an air pressure feeding system can be connected to each other. Thus, to connect the pressure feeding pad 214 with the inlets 203, it is only necessary to bring the seal member 225 attached to the pressure feeding pad 214 into contact with the upper surface 201 of the cell handling cartridge 20, thus allowing lessening of the positioning accuracy necessary for connecting the pressure feeding pad 214. Lessening the positioning accuracy can simplify the control performed by the processing circuitry 7 when the pressure feeding pad 214 and the inlets 203 are connected to each other. Lessening the positioning accuracy also allows adopting a low-cost component such as the air cylinder 213 as a pressing mechanism, thus reducing the manufacturing costs.

In addition, adopting the above connection method allows utilization of the upper surface 201 of the cell handling cartridge 20 as a contact face with the pressure feeding pad 214. Thus, it is possible to create the inlets 203 merely by forming holes in the upper surface 201. In this case, the inlets 203 need not be formed in a tubular shape matching the diameter of the seal member 225, and the diameters of the inlets 203 only need to be smaller than the diameter of the opposing face 2141 of the pressure feeding pad 214. Therefore, it is possible lessen the molding accuracy necessary when molding the inlets 203 and reduce the manufacturing costs of the cell handling cartridge 20.

### Modification of Seal Member

In the above embodiment, a case where an O-ring is used as the seal member 225 is explained; however, an elastomer other than an O-ring may be used as the seal member 225. For example, an adsorption pad may be used instead of an O-ring.

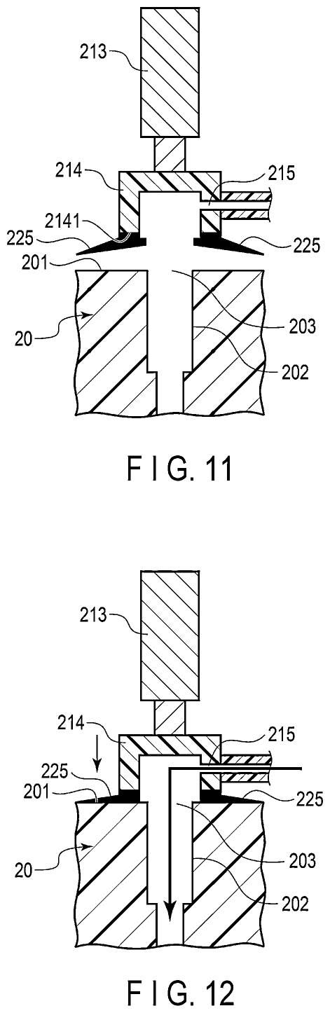

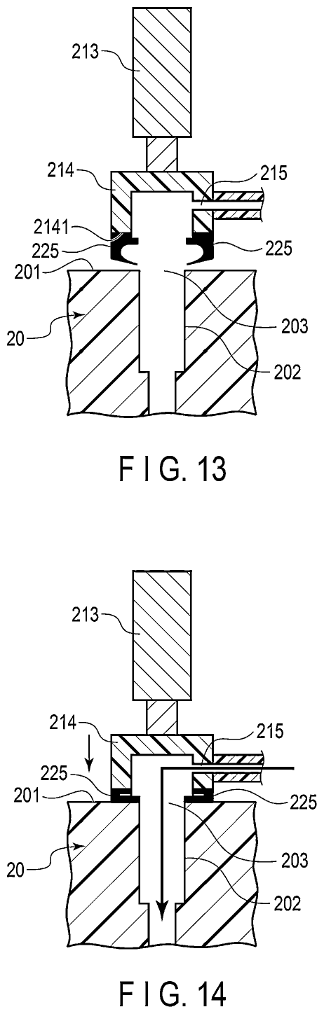

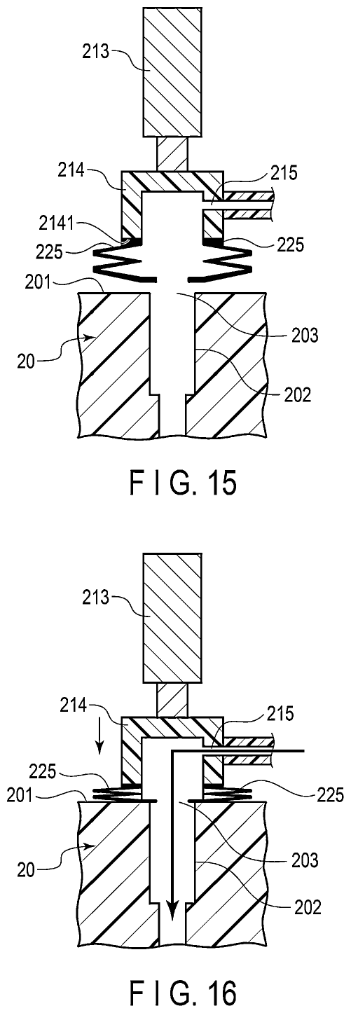

FIGS. 11 and 12 are enlarged cross-sectional views of the connection portion of the pressure feeding pad 214 and the cell handling cartridge 20 according to a first modification. FIGS. 13 and 14 are enlarged cross-sectional views of the connection portion of the pressure feeding pad 214 and the cell handling cartridge 20 according to a second modification. FIGS. 15 and 16 are enlarged cross-sectional views of the connection portion of the pressure feeding pad 214 and the cell handling cartridge 20 according to a third modification. FIGS. 11, 13, and 15 show the state in which the pressure feeding pad 214 and the inlet 203 are not connected to each other. FIGS. 12, 14, and 16 show the state in which the pressure feeding pad 214 has descended and is connected to the inlet 203. In FIGS. 11 to 16, a vacuum pad is used as the pressure feeding pad 214. In the first modification shown in FIGS. 11 and 12, an adsorption pad shaped such that its elastic portion extends outward is used as the seal member 225, and in the second modification shown in FIGS. 13 and 14, an adsorption pad shaped such that its elastic portion extends inward is used as the seal member 225. In the third modification shown in FIGS. 15 and 16, a bellows-shaped adsorption pad is used as the seal member 225.

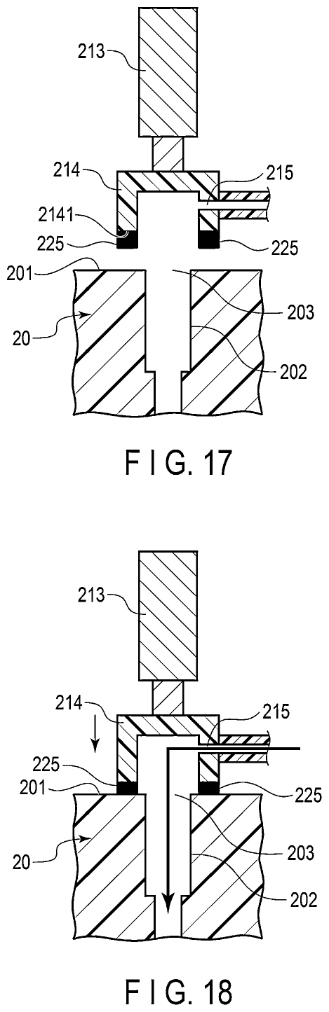

FIGS. 17 and 18 are enlarged cross-sectional views of the connection portion of the pressure feeding pad 214 and the cell handling cartridge 20 according to a fourth modification. FIG. 17 shows the state in which the pressure feeding pad 214 and the inlet 203 are not connected to each other. FIG. 18 shows the state in which the pressure feeding pad 214 has descended and is connected to the inlet 203, shifted from the state shown in FIG. 17. In this modification, a hollow flat rubber plate is used as the seal member 225. The rubber plate is attached to the entire opposing face 2141 of the pressure feeding pad 214 and provided with a hole that allows a gas to pass through.

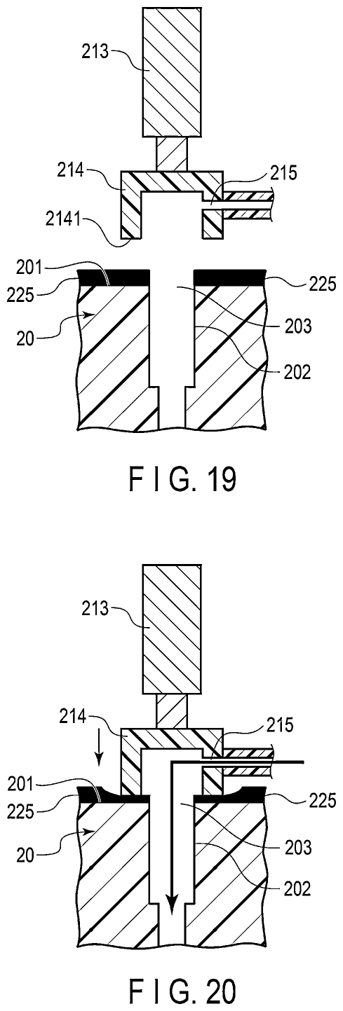

FIGS. 19 and 20 are enlarged cross-sectional views of the connection portion of the pressure feeding pad 214 and the cell handling cartridge 20 according to a fifth modification. FIG. 19 shows the state in which the pressure feeding pad 214 and the inlet 203 are not connected to each other. FIG. 20 shows the state in which the pressure feeding pad 214 has descended and is connected to the inlet 203, shifted from the state shown in FIG. 19. In this modification, the seal member 225 is on the upper surface 201 of the cell handling cartridge 20 instead of being on the opposing face 2141 of the pressure feeding pad 214. In this modification, a hollow flat rubber plate is used as the seal member 225. The rubber plate is attached to the entire upper surface 201, and the portion covering the inlet 203 is provided with a hole that allows a gas to pass through.

In this manner, the seal member 225 may be on either one of the opposing face 2141 of the pressure feeding pad 214 that faces the cell handling cartridge 20 or the opposing face (i.e., the upper surface 201) of the cell handling cartridge 20 that faces the pressure feeding pad 214. Arranging the seal member 225 on the opposing face 2141 of the pressure feeding pad 214 reduces the amount of elastomer needed, allowing for reduction of the manufacturing costs, as compared to the case of arranging the seal member 225 at each of the inlets 203 of the cell handling cartridge 20. Also, although arranging the seal member 225 at the pressure feeding pad 214 necessitates regular maintenance of the seal member 225, arranging the seal member 225 closer to the cell handling cartridge 20 that will be discarded after being used eliminates the need for regular maintenance of the seal member 225.

Also, the seal member 225 may be arranged at both the pressure feeding pad 214 and the cell handling cartridge 20.

### Second Embodiment

A second embodiment will be described. The present embodiment is a modification of the configuration of the first embodiment, as described below. Descriptions of the configuration, operation, and effect that are the same as those of the first embodiment will be omitted. In the first embodiment, the positioning of the pressure feeding pad 214 and the inlets 203 is performed by moving the pressure feeding pad 214. In the present embodiment, however, the positioning is performed by moving the cell handling cartridge 20.

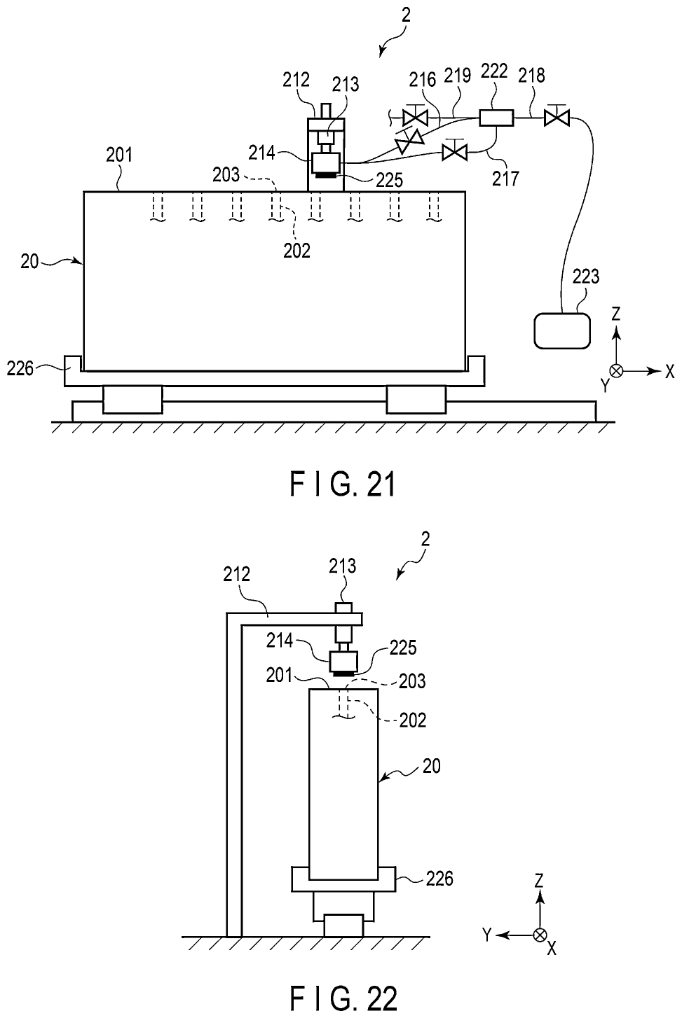

FIG. 21 is a view of the cell handling mechanism 2 according to the present embodiment from one side in the Y-axis direction. FIG. 22 is a view of the cell handling mechanism 2 from one side in the X-axis direction.

As shown in FIGS. 21 and 22, in the present embodiment, the movable support 211 is not provided with respect to the X-axis direction, and the arm 212 is fixed to the floor surface of the liquid delivery mechanism 21. Thus, the arm 212, air cylinder 213, and pressure feeding pad 214 do not move in the X-axis direction.

The cell handling mechanism 2 includes a support 226 that supports the cell handling cartridge 20. The support 226 is arranged on the bottom surface of the liquid delivery mechanism 21 and supports the cell handling cartridge 20. The support 226 is, for example, a robot that can move on the floor surface of the liquid delivery mechanism 21 in the X-axis direction. The support 226 is a moving mechanism that moves the cell handling cartridge 20 along the X-axis direction under the control of the processing circuitry 7.

Next, an operation of the cell handling system 1 of the present embodiment will be described.

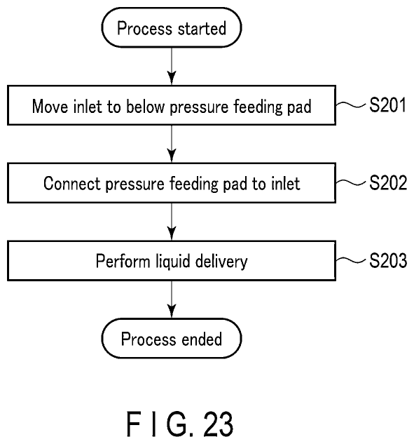

FIG. 23 is a flowchart showing an example of a procedure of liquid delivery performed by the cell handling system 1. The driving of the respective configurations described below is performed under the control of the processing circuitry 7, a description of which is omitted.

As shown in FIG. 21, the liquid delivery is started in a state where the cell handling cartridge 20 is attached to the cell handling mechanism 2 and the support 226 and the cell handling cartridge 20 are in an initial position.

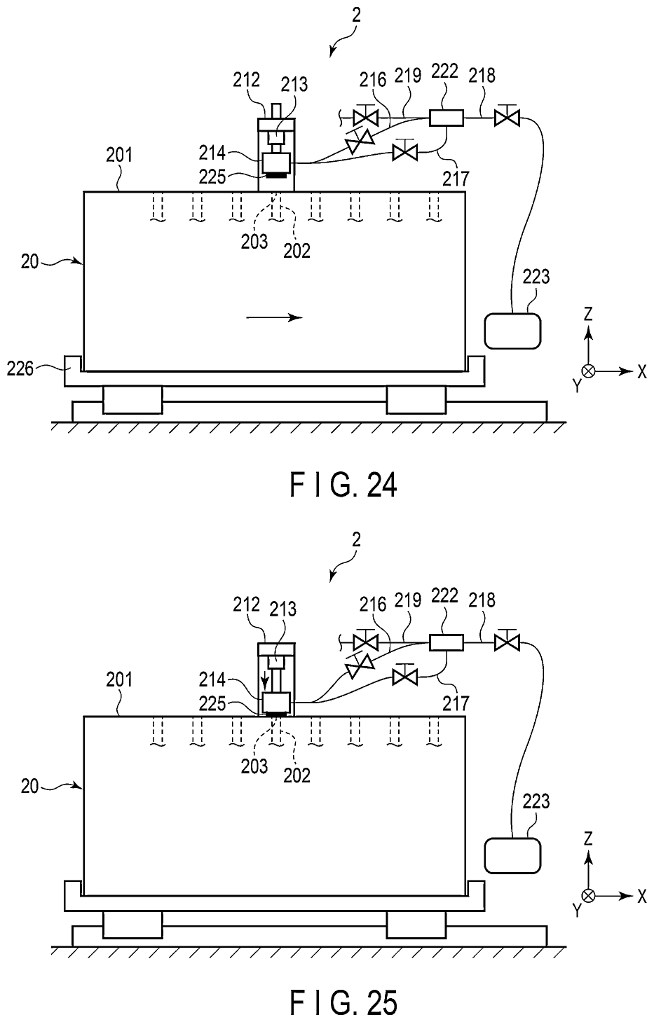

In the present embodiment, the positioning of the pressure feeding pad 214 and the cell handling cartridge 20 with respect to the horizontal direction is performed by moving the support 226 and the cell handling cartridge 20. At this time, the support 226 and the cell handling cartridge 20 are moved along the X-axis direction such that the inlet 203 through which liquid delivery is performed is positioned below the opening portion of the pressure feeding pad 214. FIG. 24 is a view of a state in which the air cylinder 213 and the pressure feeding pad 214 have moved to above the inlet 203 of a target passage. FIG. 7 is a view of the cell handling mechanism 2 from one side in the Y-axis direction.

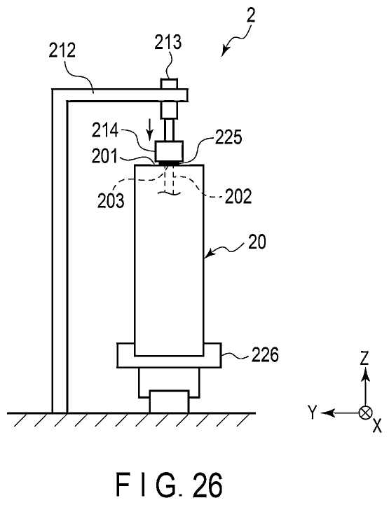

Thereafter, the pressure feeding pad 214 is connected to the inlet 203 by being pressed against the upper surface 201 of the cell handling cartridge 20 (step S202), as in step S102 of the first embodiment. FIGS. 25 and 26 show the state in which the pressure feeding pad 214 is pressed against the cell handling cartridge 20. FIG. 25 is a view of the cell handling mechanism 2 from one side in the Y-axis direction. FIG. 26 is a view of the cell handling mechanism 2 from one side in the X-axis direction.

Thereafter, liquid delivery is performed (step S203), as in step S103 of the first embodiment.

In the present embodiment as well, the seal member 225 is on the opposing face 2141 of the pressure feeding pad 214; thus, the present embodiment can also achieve the same effects as those achieved by the first embodiment.

### Third Embodiment

A third embodiment will be described. The present embodiment is a modification of the configuration of the first embodiment, as described below. Descriptions of the configuration, operation, and effect that are the same as those of the first embodiment will be omitted. The present embodiment provides a regulator for mechanically suppressing the motion of the cylinder shaft caused by a reaction to pressure feeding.

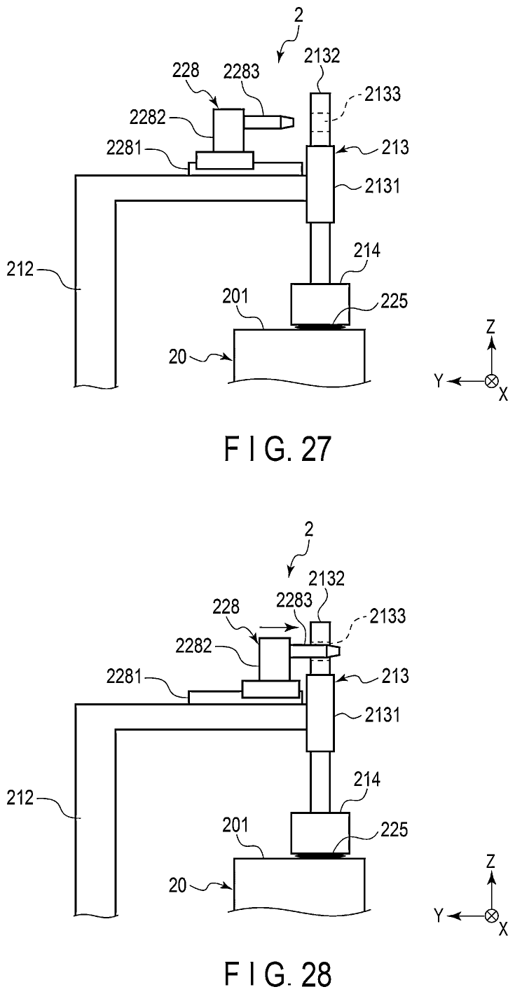

FIG. 27 is a view of the cell handling mechanism 2 according to the present embodiment from one side in the X-axis direction.

The air cylinder 213 includes a body part 2131, a cylinder shaft 2132, and an insertion hole 2133. The body part 2131 is fixed to the arm 212. The body part 2131 includes a drive mechanism inside the body part 2131 and supports the cylinder shaft 2132 so as to allow the cylinder shaft 2132 to move along the vertical direction (Z-axis direction). The pressure feeding pad 214 is fixed to a lower end of the cylinder shaft 2132. The cylinder shaft 2132 protrudes from the body part 2131 on both sides in the vertical direction.

The insertion hole 2133 is a through-hole in the cylinder shaft 2132 piercing through the cylinder shaft 2132 in the Y-axis direction. The insertion hole 2133 is in a portion of the cylinder shaft 2132 that protrudes upward from the body part 2131. The position of the insertion hole 2133 in the vertical direction (Z-axis direction) also changes in tandem with the vertical movement of the cylinder shaft 2132.

The cell handling mechanism 2 further includes a fixing mechanism 228. The fixing mechanism 228 functions as a regulator that regulates the movement of the air cylinder 213 in the direction away from the cell handling cartridge 20. The fixing mechanism 228 is fixed onto the arm 212 in a position close to the air cylinder 213.

The fixing mechanism 228 includes a support 2281, a moving object 2282, and a protruding pin 2283. The support 2281 is fixed to the arm 212. The support 2281 includes a drive mechanism inside the support 2281 and supports the moving object 2282 so as to allow the moving object 2282 to move along the Y-axis direction. The moving object 2282 moves along the Y-axis direction with respect to the support 2281 under the control of the processing circuitry 7. The protruding pin 2283 is fixed to the moving object 2282. The protruding pin 2283 protrudes from the moving object 2282 toward a distal side of the arm 212 along the Y-axis direction. That is, the protruding pin 2283 protrudes toward the air cylinder 213. In the state where the pressure feeding pad 214 is connected to the cell handling cartridge 20, the protruding pin 2283 is fixed at the height where the insertion hole 2133 of the cylinder shaft 2132 is positioned. The protruding pin 2283 is formed in a size that allows the protruding pin 2283 to be inserted through the insertion hole 2133.

FIG. 28 is a view of a state in which the moving object 2282 has moved to a distal side of the arm 212, shifted from the state shown in FIG. 27. In the state where the pressure feeding pad 214 is connected to the cell handling cartridge 20, the fixing mechanism 228 is arranged such that the protruding pin 2283 is inserted into the insertion hole 2133 when the moving object 2282 moves to the distal side of the arm 212.

The processing circuitry 7 controls the driving of the air cylinder 213, the pressure feeding device, and the fixing mechanism 228, and causes the liquid delivery to be performed by the pressure feeding device in the state where the pressure feeding pad 214 is pressed by the air cylinder 213 and in the state where the fixing mechanism 228 is connected to the air cylinder 213.

Next, an operation of the cell handling system 1 of the present embodiment will be described.

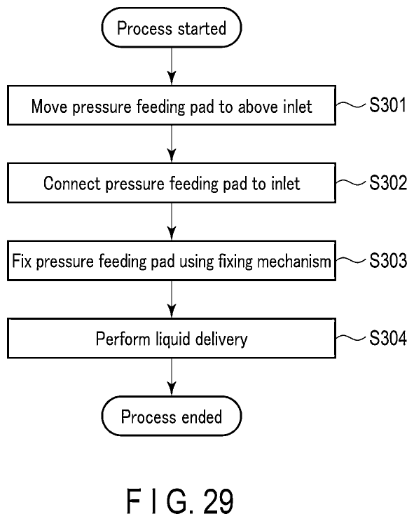

FIG. 29 is a flowchart showing an example of a procedure of liquid delivery performed by the cell handling system 1. Since steps S301 and S302 are the same as steps S101 and S102 of the first embodiment, descriptions thereof will be omitted. The driving of the respective configurations described below is performed under the control of the processing circuitry 7, a description of which is omitted.

### Step S303

As shown in FIG. 27, when the pressure feeding pad 214 is connected to the inlet 203 through which liquid delivery is performed in step S302, the moving object 2282 moves in a direction approaching the air cylinder 213 along the Y-axis direction. At this time, the protruding pin 2283 is inserted into the insertion hole 2133 of the cylinder shaft 2132 and fitted to the insertion hole 2133, as shown in FIG. 28. With the protruding pin 2283 fitted to the insertion hole 2133, the movement of the cylinder shaft 2132 with respect to the vertical direction is being regulated.

### Step S304

Next, liquid delivery is performed, as in the first embodiment. When the pneumatic pressure in the inlet 203 rises due to pressurization performed via the inlet 203, a reaction in a direction away from the cell handling cartridge 20 acts on the pressure feeding pad 214. Since the protruding pin 2283 is fitted to the insertion hole 2133 of the cylinder shaft 2132, the upward movements of the cylinder shaft 2132 and the pressure feeding pad 214 are regulated, preventing the connected state of the pressure feeding pad 214 and the inlet 203 from being released.

When the liquid delivery is completed, the moving object 2282 moves in a direction away from the air cylinder 213 along the Y-axis direction, and the protruding pin 2283 is removed from the insertion hole 2133. Then, the cylinder shaft 2132 moves upward, and the pressure feeding pad 214 is removed from the inlet 203.

Hereinafter, the effects of the cell handling system 1 according to the present embodiment will be described.

When a high-pressure gas is fed with the pressure feeding pad 214 pressed against the inlet 203, a reaction acts on the pressure feeding pad 214 and the air cylinder 213, likely causing the pressure feeding pad 214 to be pushed back, releasing the airtight state of the pressure feeding pad 214 and the inlet 203. The cell handling system 1 of the present embodiment is provided with the fixing mechanism 228 as a mechanical stopper for mechanically suppressing the motion of the cylinder shaft 2132 caused by a reaction to air-pressure liquid delivery, thereby mechanically suppressing the motion of the cylinder shaft 2132. Thus, the pressure feeding pad 214 can be prevented from being pushed back by a reaction to air-pressure liquid delivery.

Also, providing the fixing mechanism 228 can reduce the stress acting on the air cylinder 213, preventing breakage of the air cylinder 213. Further, providing the fixing mechanism 228 allows adopting a low-cost mechanism such as an air cylinder as a presser, reducing the manufacturing costs.

### Modification of Third Embodiment

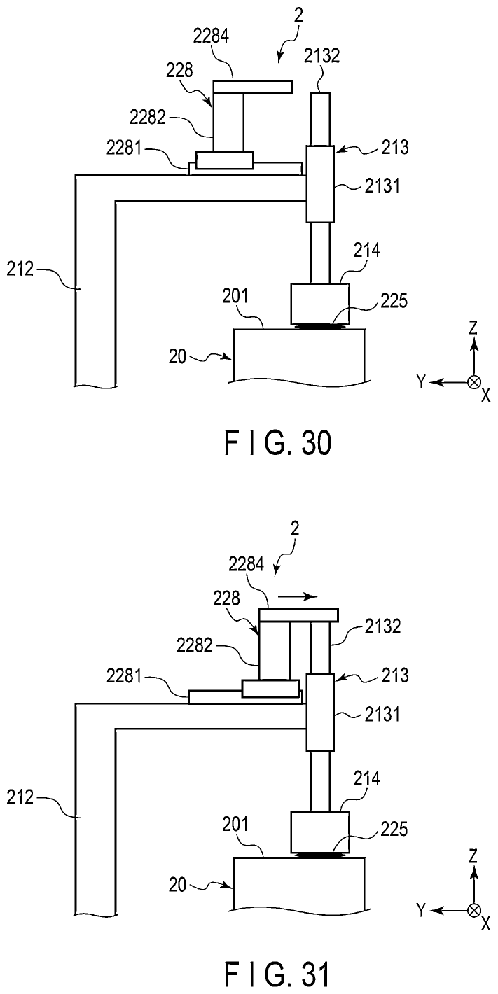

FIG. 30 is a view of the cell handling mechanism 2 according to the present modification from one side in the X-axis direction.

In the present modification, the fixing mechanism 228 includes a stopper 2284 instead of the protruding pin 2283. The stopper 2284 is fixed to the moving object 2282. The stopper 2284 protrudes from the moving object 2282 toward a distal side of the arm 212 along the Y-axis direction. That is, the stopper 2284 protrudes toward the air cylinder 213. In the state where the pressure feeding pad 214 is connected to the cell handling cartridge 20, the stopper 2284 is fixed in a position slightly higher than an upper end of the cylinder shaft 2132.

FIG. 31 is a view of a state in which the moving object 2282 has moved toward a distal side of the arm 212, shifted from the state shown in FIG. 30. In the state where the pressure feeding pad 214 is connected to the cell handling cartridge 20, the fixing mechanism 228 is arranged such that the stopper 2284 is positioned above the cylinder shaft 2132 when the moving object 2282 moves toward the distal side of the arm 212.

In the present modification, when the pressure feeding pad 214 is connected to the inlet 203 through which liquid delivery is performed, the moving object 2282 moves in a direction approaching the air cylinder 213 along the Y-axis direction. At this time, the stopper 2284 moves to above the upper end of the cylinder shaft 2132, as shown in FIG. 31. When the stopper 2284 moves to above the cylinder shaft 2132, the upward movement of the cylinder shaft 2132 is regulated, allowing the pressure feeding pad 214 to be prevented from being pushed back by a reaction to air-pressure liquid delivery.

According to at least one embodiment described above, it is possible to suppress manufacturing costs while securing airtightness in the connection between a cartridge and an air pressure feeding mechanism.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell handling system comprising:
a cell handling cartridge including a passage and an inlet, the passage configured to allow a liquid used for cell handling to flow through the passage, the inlet provided to the passage, opened toward outside, and configured to allow a gas used for delivery of the liquid to pass through the inlet;
a body apparatus including a connection portion and a presser, the connection portion being connected to the inlet and having an air passage configured to allow the gas to flow through the air passage, the presser being configured to press the connection portion against the cell handling cartridge; and
a seal member on at least one of an opposing face of the connection portion or an opposing face of the cell handling cartridge, the opposing face of the connection portion facing the cell handling cartridge, the opposing face of the cell handling cartridge facing the connection portion.

2. The cell handling system according to claim 1, wherein the seal member is formed of an elastomer.

3. The cell handling system according to claim 2, wherein the seal member is an O-ring.

4. The cell handling system according to claim 1, wherein the presser includes an air cylinder.

5. The cell handling system according to claim 1, wherein the inlet has a diameter smaller than a diameter of the opposing face of the connection portion.

6. The cell handling system according to claim 1, wherein the body apparatus further includes a regulator configured to regulate a movement of the connection portion in a direction away from the cell handling cartridge.

7. The cell handling system according to claim 6, wherein
the body apparatus includes:
a pressure feeding device connected to the connection portion and configured to deliver the liquid using compressed air; and
processing circuitry configured to control driving of the presser, the pressure feeding device, and the regulator, and
the processing circuitry causes the delivery of the liquid to be performed by the pressure feeding device with the connection portion pressed by the presser and the regulator connected to the connection portion.

8. The cell handling system according to claim 1, wherein the body apparatus includes a moving mechanism configured to hold the connection portion and horizontally move the connection portion.

9. The cell handling system according to claim 1, wherein the body apparatus includes a moving mechanism configured to hold the cell handling cartridge and horizontally move the cell handling cartridge.

10. The cell handling system according to claim 1, wherein
the body apparatus includes:
a pressure feeding device connected to the connection portion and configured to deliver the liquid using compressed air; and
processing circuitry configured to control driving of the presser and the pressure feeding device, and
the processing circuitry controls a magnitude of a force with which the connection portion presses the cell handling cartridge by controlling a force with which the presser presses the connection portion.

11. The cell handling system according to claim 1, wherein
the opposing face of the cell handling cartridge is a face perpendicular to a direction of pressing by the presser, and
the opposing face of the connection portion is a face perpendicular to the direction of pressing by the presser.

12. A cell handling apparatus comprising:
a connection portion connected to an inlet of a cell handling cartridge and having an air passage, the cell handling cartridge including a passage and the inlet, the passage configured to allow a liquid used for cell handling to flow through the passage, the inlet provided to the passage, opened toward outside, and configured to allow a gas used for delivery of the liquid to pass through the inlet, the air passage configured to allow the gas to pass through the air passage;
a presser configured to press the connection portion against the cell handling cartridge; and
a seal member on a contact face of the connection portion, the contact face coming into contact with the cell handling cartridge.

13. The cell handling apparatus according to claim 12, wherein the seal member is formed of an elastomer.

14. The cell handling apparatus according to claim 12, wherein the presser includes an air cylinder.

15. The cell handling apparatus according to claim 12, wherein the inlet has a diameter smaller than a diameter of the contact face of the connection portion.

16. The cell handling apparatus according to claim 12, further comprising a regulator configured to regulate a movement of the connection portion in a direction away from the cell handling cartridge.

17. The cell handling apparatus according to claim 16, further comprising:
a pressure feeding device connected to the connection portion and configured to deliver the liquid using compressed air; and
processing circuitry configured to control driving of the presser, the pressure feeding device, and the regulator,
wherein the processing circuitry causes the delivery of the liquid to be performed by the pressure feeding device with the connection portion pressed by the presser and the regulator connected to the connection portion.

18. The cell handling apparatus according to claim 12, further comprising:
a pressure feeding device connected to the connection portion and configured to deliver the liquid using compressed air; and
processing circuitry configured to control driving of the presser and the pressure feeding device,
wherein the processing circuitry controls a magnitude of a force with which the connection portion presses the cell handling cartridge by controlling a force with which the presser presses the connection portion.

19. The cell handling apparatus according to claim 12, wherein
the contact face of the connection portion is a face perpendicular to a direction of pressing by the presser.

20. A cell handling cartridge comprising:
a passage configured to allow a liquid used for cell handling to flow through the passage;
an inlet provided to the passage, opened toward outside, and configured to allow a gas used for delivery of the liquid to pass through the inlet;
an opposing face connected to the inlet and facing a connection portion having an air passage configured to allow the gas to flow through the air passage; and
a seal member on the opposing face.
